(19) United States
(12) Patent Application Publication
(10) Pub. No.: US 2003/0138813 A1
(43) Pub. Date: Jul. 24, 2003

(54) Method of diagnosis and disease risk assessment

(76) Inventors: Lars Engstrand, Uppsala (SE); Anders Alderborn, Uppsala (SE)

Correspondence Address: DORSEY & WHITNEY LLP, INTELLECTUAL PROPERTY DEPARTMENT, 250 PARK AVENUE, NEW YORK, NY, 10177, US

(21) Appl. No.: 10/253,055

(22) Filed: Sep. 24, 2002

Related U.S. Application Data
(60) Provisional application No. 60/324,681, filed on Sep. 25, 2001.

(30) Foreign Application Priority Data
Sep. 24, 2001 (CA) 2,357,572

(51) Int. Cl. C12Q 1/70; C12Q 1/68

(52) U.S. Cl. 435/6; 435/5

(57) ABSTRACT

This invention relates to a method of determining information about the likely clinical outcome of a microbiological infection in a patient, and also to a method of selecting a suitable therapeutic regimen for a patient with a microbiological infection.

Accordingly, in one aspect, the present invention provides a method of determining the likely clinical outcome of a microbiological infection in a patient comprising:

(a) analysing a sample taken from said patient for the presence of a target micro-organism, by the characterisation of a target nucleic acid sequence therein; and

(b) analysing a sample taken from said patient for the presence of one or more disease susceptibility markers in the genome of said patient.

Alternatively viewed, in another aspect, the present invention provides a method of selecting a suitable therapeutic regimen for a patient comprising:

(a) analysing a sample taken from said patient for the presence of a target micro-organism, by the characterisation of a target nucleic acid sequence therein; and

(b) analysing a sample taken from said patient for the presence of one or more disease susceptibility markers in the genome of said patient.

### CROSS REFERENCE TO RELATED APPLICATION

This application claims the benefit of U.S. Application Serial No. 60/324,681 filed Sep. 25, 2001, the disclosure of which is incorporated herein by reference.

### BACKGROUND OF THE INVENTION

This invention relates to a method of determining information about the likely clinical outcome of a microbiological infection in a patient, and also to a method of selecting a suitable therapeutic regimen for a patient with a microbiological infection.

Microbiological infections, i.e. infection of a host organism by micro-organisms, are one of the major causes of morbidity in general populations. In order to treat patients effectively, the infection must be diagnosed and an appropriate therapeutic regimen used. It is of great clinical importance to identify (“type”) the micro-organism involved in the infection as then the disease can be properly diagnosed.

Conventional methods of typing microbiological infections involve culturing a sample taken from the patient (e.g. blood sample), and re-culturing on selective growth medium. Biochemical characterization of the micro-organism involved may then take place. Suitable methods of biochemical characterization include gram staining, colonial morphology, indole production testing and O—F reaction (testing whether an organism utilises glucose fermentivatively, oxidatively, or not at all). These assays result in the identification of the species of micro-organism involved in the infection, and provide no further information regarding the infection. The problems with conventional methods of typing micro-organisms are multiple and can severely hinder prompt diagnosis of infection. Culturing micro-organisms can be time-consuming, especially when the organism is slow growing or even non-cultivatable. For newer species there is a lack of accurate methods for typing.

Identifying the micro-organism involved in the infection is an important step in determining the correct treatment for the infection. In epidemiology, species information is also extremely important to determine the source and mode of transmission.

Classical identification methods based on biochemical, serological, morphological and phenotypic characteristics are traditionally used to identify micro-organism infections. However, as more information becomes available regarding micro-organisms at the genetic level, the emphasis of diagnostic studies is shifting towards molecular methods such as sequencing of the 16S rRNA genes of bacteria. One advantage of molecular biology based identification or typing of micro-organisms is that there is no need to culture samples. However, conventional sequencing methods used for typing (such as pulse field electrophoresis, hybridization or gel-based sequencing) can be time consuming, days or weeks may be required, and some methods are difficult to perform. It is thus an object of the present invention to offer accurate and quick nucleic acid analysis and hence diagnosis of the infection. Such information is vital, especially with life-threatening infections and epidemics of infection.

Identification of the species of micro-organism involved in the infection does not provide all the information required for the diagnosis, treatment and/or prognosis of the infection in the patient.

For accurate diagnosis, it would be advantageous not only to determine the general “class” (or genus or species) of infecting micro-organism present, but also to determine which of the sub-types (e.g. strains) is present. For many infections, e.g. viral infections such as hepatitis C infection, the infecting micro-organism may occur in a number of different sub-types (strains or genotypes), for example seven sub-types (or genotypes) are known of the HCV virus. The advantage of using molecular biology based techniques is that the sub-type (strain or genotype) of the infection micro-organism can be identified. Molecular biology based analysis of the micro-organism involved in the infection thus offers some advantages over standard techniques.

The virulence or pathogenicity of micro-organisms such as bacteria and viruses depend upon their ability to multiply in the host. “Virulence genes” are those genes which are involved in the regulation of virulence during infection. Virulence genes may thus be defined as genes whose products are involved in interactions with the host and are responsible for pathological damage. For example in Vibrio cholerae the Cholera toxin is the virulence factor which is primarily responsible for the disease symptom, severe diarrhoea, and several virulence genes are involved in the expression of this virulence factor. The virulence factors are apparently required at various times to cause disease. The presence of certain virulence genes can be associated with enhanced virulence, and is it therefore important to identify what virulence genes are present. Screening for the presence of virulence markers could prove useful in infection control.

Another factor which may be considered in the identification of the causative micro-organism of infection is the drug-resistance of the micro-organism. For example antibiotic resistant bacterial strains are becoming more prevalent, and multi-drug resistant strains are emerging. Drug resistance in micro-organisms result from drug resistance genes and modified structural genes (e.g. modified cell wall components). The indiscriminate use of antibiotic drugs against infections may increase the number of antibiotic-resistant bacteria and therefore, rationalized use of antibiotics should be made. In diagnosing patients with an infection, it would be helpful to determine the drug-resistance of the strain involved, as then the treatment regimen given can avoid the drugs to which the strain is resistant. Drug-resistant infections can sometimes increase the risk of death, and are often associated with prolonged illness and related complications. Early identification of such drug-resistant infections and determination of the correct treatment is therefore vital.

The genetics of the patient or host organism can also be important in making a complete prognosis of the clinical outcome of the infection. For example, the host genes can influence the differential susceptibility of individuals or populations to infectious diseases. Genes have been identified in the human genome that modify the infectious disease risk, such as variants of vitamin D receptor genes influencing the susceptibility to tuberculosis (TB) and other mycobacterial diseases. Micro-organism infections can lead to the development of a related secondary disease within the host organism. For example Helicobacter pylori infection is associated with a variety of clinical outcomes including peptic ulcer disease and the development of gastric cancer. Host genetic risk factors for developing these diseases have been identified (El-Omar et al, Nature 2000, Vol. 404, 398 to 402). Specific types of the human papillomaviruses (HPV) play a casual role in cervical factor. However, few women infected with HPV progress to cervical carcinoma, and therefore the genetic make up of the patient may influence susceptibility to developing carcinoma.

Other host susceptibility factors that can be identified at the genetic level include whether they will respond to a particular drug or not. For example, whether the patient has an enzyme necessary to convert a prodrug into an active drug to combat the infection.

Therefore, it can be seen that rapid identification of micro-organism sub-type, virulence factors and/or drug resistance and obtaining host risk-factor information is important in making decisions on an optimal therapeutic regimen and provides a much more complete picture of the infection that identification of the micro-organism alone. Conventional biotyping to determine species identity, drug resistance status and level of virulence is time-consuming and involves an array of assays, including biochemical and microbial culturing techniques. Further, such methods give no information on the host and their genetic risk factors such as inability to metabolise drugs or predisposition to a secondary disease.

There is thus a need to provide fuller information on the likely clinical outcome of a microbiological infection in a patient quickly and accurately. The present invention addresses this need.

### SUMMARY OF THE INVENTION

In particular, it has now been found that a rapid, reliable and accurate method for obtaining information on the likely clinical outcome of a microbiological infection in a patient can be obtained by analysing and/or characterising a plurality of nucleic acid molecules from a sample or samples taken from said patient.

This new method of the invention thus provides clinically relevant information about the micro-organism and clinically relevant information about the patient. This information can then be combined, allowing the clinician to predict the likely outcome of the infection.

The method is also particularly suited to selecting a suitable therapeutic regimen for a patient using the information about infectious agent and the patient obtained by characterisation of target nucleic acid sequences within the genomes of the host and the micro-organism.

### BRIEF DESCRIPTION OF THE DRAWINGS

FIG. 1 depicts a segment of the 16S rRNA gene of H. pylori, and the H. pylori specific sequences CGCGCAATCAGCGTCAGTAA which can be used to indicate the presence of H. pylori in a sample;

FIG. 2 depicts a segment of the 23S rRNA gene of H. pylori. Positions 2142 and 2143 are marked and the possible nucleotides at these positions are shown. If the polymorphic pattern at these residues is AA the H. pylori is sensitive to the antibiotic clarithromycin. If the polymorphic pattern GA or AG is present, the H. pylori is resistant to clarithromycin;

FIG. 3 depicts a segment of the H. pylori genome containing the cag pathogenesis island (cag PA1). The cag A gene is a marker for cag PA1. Possible positioning of PCR and sequencing primers (arrows) are shown;

FIG. 4 is a trace (light intensity on Y-axis versus nucleotide addition on X-axis) obtained from a DNA sequencing reaction on the 16S rRNA gene from H. pylori (see Example 1);

FIG. 5 is a trace (light intensity versus nucleotide addition) obtained from a DNA sequencing reaction on the 23S rRNA gene of H. pylori. These results show the polymorphic pattern (“the marker”) is AA and thus the H. pylori is sensitive to clarithromycin;

FIG. 6 is a trace (light intensity versus nucleotide addition) obtained from a DNA sequencing reaction on the 23S rRNA gene of H. pylori. These results show the polymorphic pattern (“the marker”) is AG and thus the H. pylori is resistant to clarithromycin;

FIG. 7 is a trace (light intensity versus nucleotide addition) obtained from a DNA sequencing reaction on the 23S rRNA gene of H. pylori. These results show the polymorphic pattern (“the marker”) is GA and thus the H. pylori is resistant to clarithromycin;

FIG. 8 is a series of traces (light intensity verus nucleotide addition) obtained from DNA sequencing reactions on the patient genome, looking at interleukin-1-beta position −511.

FIG. 8a depicts the trace obtained for the heterozygote C/T;

FIG. 8b depicts the trace obtained for the heterozygote C/C;

FIG. 8c depicts the trace obtained for the heterozygote T/T, which is associated with a higher risk of developing gastric cancer associated with H. pylori infection.

FIG. 9 is a trace (light intensity versus nucleotide addition) obtained from a DNA sequencing reaction on the Cag A gene of H. pylori. These results show the presence of the Cag A gene associated with higher risk of peptid ulcer disease, atrophic gastritis and gastric cancer.

### DETAILED DESCRIPTION OF THE INVENTION

Accordingly, in one aspect, the present invention provides a method of determining the likely clinical outcome of a microbiological infection in a patient comprising:

(a) analysing a sample taken from said patient for the presence of a target micro-organism, by the characterisation of a target nucleic acid sequence therein; and

(b) analysing a sample taken from said patient for the presence of one or more disease susceptibility markers in the genome of said patient.

Alternatively viewed, in another aspect, the present invention provides a method of selecting a suitable therapeutic regimen for a patient comprising:

(a) analysing a sample taken from said patient for the presence of a target micro-organism, by the characterisation of a target nucleic acid sequence therein; and

(b) analysing a sample taken from said patient for the presence of one or more disease susceptibility markers in the genome of said patient.

In both methods, the information obtained in steps (a) and (b) is then used to determine the likely clinical outcome and/or to select an appropriate therapeutic regimen.

The term “clinical outcome” as used herein includes all the possible consequences of a microbiological infection of a patient (e.g. complete recovery, extended illness, contracting/developing secondary related disease or morbidity). As described above, the clinical outcome of an infection is multifactorial, it relies upon numerous factors including which micro-organism is involved in the infection, which particular strain is present, how virulent the micro-organism is, how resistant to drugs the micro-organism is and whether the host (patient) possesses disease susceptibility markers. The clinical outcome will depend also on the treatment regimen administered, but when the information about the micro-organism and the host is first assessed, the prognosis (determination of clinical outcome) will typically give the likely outcome assuming no therapeutic or other unexpected intervention.

“Therapeutic regimen” according to the present invention can involve any method of treatment which is directed either towards eliminating or controlling the infectious micro-organism and/or to dealing with the symptoms or possible secondary effects of the infection. The regimen will be ‘suitable’ having regard to the factors investigated by the method of the invention. It is envisaged that suitable therapeutic regimens would include the administration of one or more drugs (or pharmaceutical composition, medication or prophylactic), but may also extend to other therapeutic methods such as surgery, alteration of diet, exercise and/or gene therapy. In some instances, the most appropriate treatment regimen may be no treatment, for example if only a non-virulent strain is present. In the method of the invention, the therapeutic regimen can be prescribed for an individual patient with a microbiological infection, the regimen is thus individually tailored and highly specific.

“Microbiological infection” as used herein refers to attachment and/or invasion and typically multiplication of micro-organisms in body tissues and/or fluids. Micro-organisms capable of producing an infection include bacteria, viruses, fungi, mycoplasma and protozoa. Micro-organisms include any organism too small to be visible to the naked eye. A micro-organism will be a ‘target’ micro-organism in that a method is selected which enables that micro-organism to be differentiated from other non-target micro-organisms. For example it would rarely be appropriate to analyse a sample simply for the presence of bacteria generally and the target bacteria would be a particular class, species or sub-species etc. The target micro-organism typically being responsible or potentially responsible for undesirable symptoms or secondary complications in the patient. The ‘target’ micro-organism being distinguished from other non-target micro-organisms on the basis of the presence or character of a target nucleic acid sequence found in said micro-organism.

The “patient” may be human, or a veterinary patient, such as farm animals including cattle, horses, sheep, pigs or chickens, companion animals such as dogs and cats, primates such as chimpanzees and gorillas, or any other animal. Herein, the term animal includes fish and birds.

As used herein a “sample” refers to any suitable sample or specimen that can be taken from a patient to determine the presence of a microbiological infection. It will be appreciated by the person skilled in the art that a suitable sample will be taken for a particular infection e.g. urine sample for suspected kidney infections. Suitable samples include body fluids, i.e. blood, serum, lymph, urine, spinal fluid, saliva or semen. Other samples are also suitable and include biopsy samples; e.g. skin, gastric biopsy, rectal; vaginal; buccal or wound swabs and faeces samples.

As used herein “nucleic acid” may be any nucleic acid, it may be DNA, RNA (e.g. mRNA) or any derivative thereof. If it is desired to type a RNA sample, the method may additionally include the step of generating cDNA from the RNA template, conveniently by using reverse transcriptase. Alternatively, if desired, the characterization of the nucleic acid may be performed directly on the RNA molecule(s).

In the method of the invention, characterisation of a target nucleic acid sequence present in the sample takes place. The presence or nature of that sequence being indicative of the presence of the target micro-organism in the sample and thus, it is assumed, in the patient. Of course, the method may be performed for the purposes of diagnosis and the sample may not always contain the target micro-organism/nucleic acid sequence. Alternatively, target and non-target micro-organisms may have a target nucleic acid sequence which is analysed, the character of the sequence differentiating between target and non-target micro-organisms.

Any suitable method of characterising the target nucleic acid can be used in the method of the invention, and include, but are not limited to the following: specific probe hybridisation (e.g. using fluorescently labelled probes and/or radio-labelled probes) and sequencing methodologies such as Maxam-Gilbert and capillary array electrophoresis. Many methods of sequencing nucleic acids exist and many are based on an enzymatic procedure to synthesize complementary nucleic acid chains. Such sequencing methods generally rely upon a polymerase enzyme and a sequencing primer to generate a complementary strand or strands to the single-stranded template nucleic acid. The methodologies differ in how the incorporation of a base into the complementary strand is detected. The Sanger method employs dideoxy nucleotides (ddNTPs) causing complementary chain termination and the sequence is determined by size fractionation of the product in a gel or by mass spectrometry. Variations of this method use radioactively or fluorescently labelled nucleotides or dideoxy nucleotides.

A preferred method of sequencing is “sequencing-by-synthesis” (see e.g. U.S. Pat. No. 4,863,849 of Melamede). This is a term used in the art to define sequencing methods which rely upon the detection of nucleotide incorporation during a primer-directed polymerase extension reaction. The four different nucleotides (i.e. A, G, T or C nucleotides) are added cyclically or sequentially (conveniently in a known order), and the event of incorporation can be detected in various ways, directly or indirectly. This detection reveals which nucleotide has been incorporated, and hence sequencing information; when the nucleotide (base) which forms a pair (according to the normal rules of base pairing, A-T and C-G) with the next base in the template target sequence is added, it will be incorporated into the growing complementary strand (i.e. the extended primer) by the polymerase, and this incorporation will trigger a detectable signal, the nature of which depending upon the detection strategy selected.

In carrying out the invention as defined above, a sample taken from a patient is analysed for the presence of a target micro-organism by the characterisation of a target nucleic acid sequence. It will be understood that the presence or character of the target nucleic acid sequence chosen will be indicative of the target micro-organism, e.g. it will be a sequence not present in that form in other micro-organisms, i.e. a “signature sequence”. For example, the target nucleic acid to be characterised can be the 16S rRNA or the RNase P gene. A suitable signature or target sequence may be species or even sub-species specific or it may be common to a target group or class of micro-organisms. For example, the target nucleic acid to be characterised for Listeria monocytogenes is within the inLB gene.

In a preferred aspect of the invention, the nucleic acid of said target micro-organism is further analysed for the presence of drug resistant markers and/or virulence markers. Thus, further target nucleic acid sequences of the target micro-organism are characterised.

A preferred method for determining the likely clinical outcome of a microbiological infection in a patient or selecting a suitable therapeutic regimen further comprises:

(c) analysing a sample taken from said patient for the presence of one or more drug resistance markers in the genome of the target micro-organism; and/or

(d) analysing a sample taken from said patient for the presence of one or more virulence markers in the genome of the target micro-organism.

As discussed previously, the drug resistance and virulence of a micro-organism can be central to the determination of likely clinical outcome. Thus, to enable a more complete picture of the likely progression of the infection and determine optimal therapeutic regimens, information about the virulence and/or drug resistance of the micro-organisms may be obtained. For example, the virulence gene in the bacterium Mycobacterium tuberculosis has been identified. This bacteria is responsible for tuberculosis (TB). The virulence gene is called erp, and it is therefore possible to analyse the nucleic acid of Mycobacterium tuberculosis if it is the target micro-organism for the presence of a virulence marker (e.g. a specific sequence present only in the erp gene). A further example is described herein where a region of the 23S rRNA gene in H. pylori is characterised (sequenced) to determine clarithromycin resistance.

In the method of the invention the sample from the patient is analysed for the presence of one or more disease susceptibility markers in the genome of the patient. The disease susceptibility markers can be any suitable marker in the genome, and may include any one or more of the following: mutations, allelic variants, inversions, duplications, multiplications, translocations, deletions and/or insertions. Typically, the marker may be a single nucleotide polymorphism (SNP). The disease susceptibility marker may be any marker associated with, or pre-disposing to, a disease, or it may be a particular factor or feature, e.g. a genetic factor or feature (e.g. blood group or HLA genotype etc.) or a physiological factor or feature (such as gastric acid secretion) present in the host which may be associated with, or which may predispose to, or which may be a risk factor for a disease, condition, syndrome or illness. For example, host acid gastric physiology has been found to affect the clinical outcome of H. pylori infections. The sample may be analysed for the presence of one or more disease susceptibility markers by any suitable means, including any means for scanning genes for single nucleotide polymorphisms (SNPs), point mutations, deletions, insertions, or any allelic variations. Suitable methods include sequencing, mini-sequencing, PCR using allele-specific primers (ARMS rest), Taq man, oligonucleotide ligation assay (OLA) and/or allele-specific oligonucleotide hybridization (ASO).

As used herein “markers” in the nucleic acid or genome refer to a single nucleotide, multiple nucleotides, or region in the nucleic acid, the presence, absence or character of which determines the phenotype of the micro-organism or patient in relation to the trait analysed (e.g. drug resistance, virulence or disease susceptibility). Analysis of the marker may be performed by any suitable means as hereinbefore described.

The “disease susceptibility” markers in the genome of said patient include any marker in the genome which indicates the patient's response to the microbial infection, their susceptibility to or risk of developing a secondary related disease or condition (e.g. cancer) or their ability to metabolise relevant therapeutic drugs. For example, polymorphisms in the human Interleukin-1-Beta gene are thought to increase the risk of gastric cancer induced by H. pylori. The invention particularly relates to those markers which relate to the risk of associated secondary diseases.

Thus, a patient may have a disease susceptibility marker which is indicative of an adverse reaction to infection which is not exhibited by all those infected. For example, it is known that patients respond differently to group A streptococcal infections and HIV infections.

The presence of a target micro-organism is determined using the methods of the invention. It forms a preferred aspect of the invention that the strain or sub-type of micro-organism is also identified. Thus, not only is the presence of a species of micro-organism determined, but the specific strain or sub-type also identified. Strains or sub-types can vary slightly from each other in many different ways. Several hundred strains of each species of micro-organism may exist, and it may therefore be important to identify which strain is involved in an infection prior to determining likely clinical outcome or optimal therapeutic regimen.

In the method of the invention a sample (or samples) is taken from the patient. Typically the sample analysed in steps (a) and (b) are taken from the same tissue or body fluid and can therefore be prepared for subsequent nucleic acid analysis in the same way. Preferably, the samples are gathered during the same procedure and most preferably steps (a) and (b) are performed on a single sample taken from said patient. Thus use of a single gastric biopsy, throat (mouth) swab, skin biopsy/sample or CNS fluid sample is particularly preferred. When other markers (i.e. virulence and/or drug resistance) are also analysed, preferably the analysis is performed on the same single sample taken from said patient. Thus the invention offers significant benefits in terms of the discomfort suffered by the patient and the convenience and speed of analysis while providing accurate information about both the infectious agent and the host.

In a preferred embodiment of the invention, the nucleic acid of the micro-organism and patient is analysed or characterised by sequencing. In a further preferred embodiment, the sequencing is performed by sequencing-by-synthesis, wherein any suitable means for detecting incorporation of nucleotides is used such as by incorporation of labelled activated nucleotides which may subsequently be detected, or by using labelled probes which are able to bind to the extended sequence. Further detection methods are disclosed extensively in U.S. Pat. No. 863,849, e.g. spectrophotometrically or by fluorescent detection techniques, for example by determining the amount of nucleotide remaining in the added nucleotide feedstock, following the nucleotide incorporation step.

In a sequencing-by-synthesis reaction, determination of the pattern of nucleotide incorporation occurs simultaneously with primer extension. The “primer extension” reaction includes all forms of template-directed polymerase-catalysed nucleic acid synthesis reactions. Conditions and reagents for primer extension reactions are well known in the art, and any of the standard methods, reagents and enzymes etc. may be used in this step (see e.g. Sambrook et al., (eds), Molecular Cloning: a laboratory manual (1989), Cold Spring Harbor Laboratory Press). Thus, the primer extension reaction at its most basic, is carried out in the presence of primer, deoxynucleotides (dNTPs) and a suitable polymerase enzyme e.g. T7 polymerase, Klenow or Sequenase Ver 2.0 (USB USA), or indeed any suitable available polymerase enzyme. As mentioned above, for an RNA template, reverse transcriptase may be used. Conditions may be selected according to choice, having regard to procedures well known in the art.

The primer is thus subjected to a primer-extension reaction in the presence of a nucleotide, whereby the nucleotide is only incorporated if it is complementary to the base immediately adjacent (3′) to the primer position. The nucleotide may be any nucleotide capable of incorporation by a polymerase enzyme into a nucleic acid chain or molecule. Thus, for example, the nucleotide may be a deoxynucleotide (dNTP, deoxynucleoside triphosphate) or dideoxynucleotide (ddNTP, dideoxynucleoside triphosphate). Thus, the following nucleotides may be used in the primer-extension reaction: guanine (G), cytosine (C), thymine (T) or adenine (A) deoxy- or dideoxy-nucleotides. Therefore, the nucleotide may be dGTP (deoxyguanosine triphosphate), dCTP (deoxycytidine triphosphate), dTTP (deoxythymidine triphosphate) or DATP (deoxyadenosine triphosphate). As discussed further below, suitable analogues of DATP, and also for dCTP, dGTP and dTTP may also be used. Dideoxynucleotides may also be used in the primer-extension reaction. The term “dideoxynucleotide” as used herein includes all 2′-deoxynucleotides in which the 3′ hydroxyl group is modified or absent. Dideoxynucleotides are capable of incorporation into the primer in the presence of the polymerase, but cannot enter into a subsequent polymerisation reaction, and thus function as a “chain terminator”.

If the nucleotide is complementary to the target base, the primer is extended by one nucleotide, and inorganic pyrophosphate is released. As discussed further below, in a preferred method, the inorganic pyrophosphate may be detected in order to detect the incorporation of the added nucleotide.

One working definition of Sequencing by synthesis is a method in which a single activated (i.e. labelled) nucleotide is or is not incorporated into a primed template, incorporation being detected by any suitable means. This step is repeated by addition of a different activated nucleotide and incorporation is again detected. These steps are repeated and from the sum of incorporated nucleic acids the sequence can be deduced. The preferred method of sequencing-by-synthesis is however a pyrophosphate detection-based method.

Preferably, therefore, nucleotide incorporation is detected by detecting PPi release, preferably by luminometric detection, and especially by bioluminometric detection.

PPi can be determined by many different methods and a number of enzymatic methods have been described in the literature (Reeves et al., (1969), Anal. Biochem., 28, 282-287; Guillory et al., (1971), Anal. Biochem., 39, 170-180; Johnson et al., (1968), Anal. Biochem., 15, 273; Cook et al., (1978), Anal. Biochem. 91, 557-565; and Drake et al., (1979), Anal. Biochem. 94, 117-120).

It is preferred to use luciferase and luciferin in combination to identify the release of pyrophosphate since the amount of light generated is substantially proportional to the amount of pyrophosphate released which, in turn, is directly proportional to the amount of nucleotide incorporated. The amount of light can readily be estimated by a suitable light sensitive device such as a luminometer. Thus, luminometric methods offer the advantage of being able to be quantitative.

Luciferin-luciferase reactions to detect the release of PPi are well known in the art. In particular, a method for continuous monitoring of PPi release based on the enzymes ATP sulphurylase and luciferase has been developed (Nyren and Lundin, Anal. Biochem., 151, 504-509, 1985; Nyren P., Enzymatic method for continuous monitoring of DNA polymerase activity (1987) Anal. Biochem Vol 167 (235-238)) and termed ELIDA (Enzymatic Luminometric Inorganic Pyrophosphate Detection Assay). The use of the ELIDA method to detect PPi is preferred according to the present invention. The method may however be modified, for example by the use of a more thermostable luciferase (Kaliyama et al., 1994, Biosci. Biotech. Biochem., 58, 1170-1171) and/or ATP sulfurylase (Onda et al., 1996, Bioscience, Biotechnology and Biochemistry, 60:10, 1740-42). This method is based on the following reactions:

| | | |
|---|---|---|
| | ATP sulphurylase | |
| PPi + APS | → | ATP + SO₄²⁻ |
| | luciferase | |
| ATP + luciferin + O₂ | → | AMP + PPi + oxyluciferin + |
| | | CO₂ + hv |

(APS = adenosine 5′-phosphosulphate)

Reference may also be made to WO 98/13523 and WO 98/28448, which are directed to pyrophosphate detection-based sequencing procedures, and disclose PPi detection methods which may be of use in the present invention.

In a PPi detection reaction based on the enzymes ATP sulphurylase and luciferase, the signal (corresponding to PPi released) is seen as light. The generation of the light can be observed as a curve known as a Pyrogram™. Light is generated by luciferase action on the product, ATP (produced by a reaction between PPi and APS (see below) mediated by ATP sulphurylase) and, where a nucleotide-degrading enzyme such as apyrase is used, this light generation is then “turned off” by the action of the nucleotide-degrading enzyme, degrading the ATP which is the substrate for luciferase. The slope of the ascending curve may be seen as indicative of the activities of DNA polymerase (PPi release) and ATP sulphurylase (generating ATP from the PPi, thereby providing a substrate for luciferase). The height of the signal is dependent on the activity of luciferase, and the slope of the descending curve is, as explained above, indicative of the activity of the nucleotide-degrading enzyme. As explained below in the context of a homopolymeric region, peak height is also indicative of the number of nucleotides incorporated for a given nucleotide addition step. Then, when a nucleotide is added, the amount of PPi released will depend upon how many nucleotides (i.e. the amount) are incorporated, and this will be reflected in the slope height.

Advantageously, by including the PPi detection enzyme(s) (i.e. the enzyme or enzymes necessary to achieve PPi detection according to the enzymatic detection system selected, which in the case of ELIDA, will be ATP sulphurylase and luciferase) in the polymerase reaction step, the method of the invention may readily be adapted to permit extension reactions to be continuously monitored in real-time, with a signal being generated and detected, as each nucleotide is incorporated.

Thus, the PPi detection enzymes (along with any enzyme substrates or other reagents necessary for the PPi detection reaction) may simply be included in the polymerase reaction mixture.

A potential problem which has previously been observed with PPi-based sequencing methods is that DATP, used in the chain extension reaction, interferes in the subsequent luciferase-based detection reaction by acting as a substrate for the luciferase enzyme. This may be reduced or avoided by using, in place of deoxyadenosine triphosphate (ATP), a DATP analogue which is capable of acting as a substrate for a polymerase but incapable of acting as a substrate for a PPi-detection enzyme. Such a modification is described in detail in WO98/13523.

The term “incapable of acting” includes also analogues which are poor substrates for the detection enzymes, or which are substantially incapable of acting as substrates, such that there is substantially no, negligible, or no significant interference in the PPi detection reaction.

Thus, a further preferred feature of the invention is the use of a DATP analogue which does not interfere in the enzymatic PPi detection reaction but which nonetheless may be normally incorporated into a growing DNA chain by a polymerase. By “normally incorporated” is meant that the nucleotide is incorporated with normal, proper base pairing. In the preferred embodiment of the invention where luciferase is a PPi detection enzyme, the preferred analogue for use according to the invention is the [1-thio]triphosphate (or -thiotriphosphate) analogue of deoxy ATP, preferably deoxyadenosine [1-thio]triphospate, or deoxyadenosine-thiotriphosphate (DATP S) as it is also known. DATP S, along with the -thio analogues of dCTP, dGTP and dTTP, may be purchased from Amersham Pharmacia. Experiments have shown that substituting dATP with DATP S allows efficient incorporation by the polymerase with a low background signal due to the absence of an interaction between DATP S and luciferase. False signals are decreased by using a nucleotide analogue in place of DATP, because the background caused by the ability of DATP to function as a substrate for luciferase is eliminated. In particular, an efficient incorporation with the polymerase may be achieved while the background signal due to the generation of light by the luciferin-luciferase system resulting from DATP interference is substantially decreased. The dNTP S analogues of the other nucleotides may also be used in place of the other dNTPs.

Another potential problem which has previously been observed with sequencing-by-synthesis methods is that false signals may be generated and homopolymeric stretches (i.e. CCC) are difficult to sequence with accuracy. This may be overcome by the addition of a single-stranded nucleic acid binding protein (SSB) once the extension primers have been annealed to the template nucleic acid. The use of SSB in sequencing-by-synthesis is discussed in WO 00/43540 of Pyrosequencing AB.

In a preferred embodiment of the invention the extension primers are designed to bind at or near to the markers (e.g. virulence, drug resistance or host susceptibility) and at or near to the signature sequence of the micro-organism.

This allows for the analysis of the marker or the characterization of the signature sequence to be performed quickly. It will be understood that the analysis or characterisation of each nucleic acid will take place individually.

The principles and benefits of the methods of the invention can be further explained with reference to H. pylori, which has been implicated in the development of gastric cancer. H. pylori is a gastric bacterium which causes gastritis and duodenal ulcers and is associated with gastric cancer. 50% of the population of the western world is infected by H. pylori, 10% of which have developed ulcers. Current diagnostic techniques for infection with this bacteria include urea breath tests, PCR and serological tests. Direct detection of the bacteria is done by culturing gastric biopsy specimens or by histological examination of stained tissue. In Example 1, it is shown that information on the likely clinical outcome of infection by H. pylori can be obtained by looking at four factors: species identity, resistance to common antibiotics markers, virulence markers and host susceptibility markers.

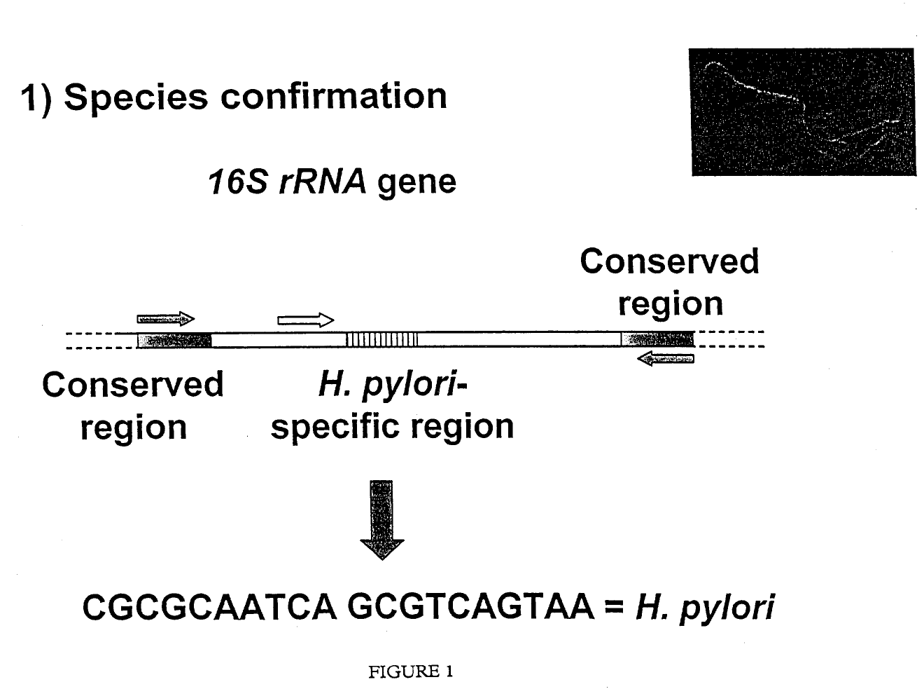

To confirm identify of the micro-organism a 20 base pair stretch of the 16S rRNA gene is sequenced, see FIG. 1.

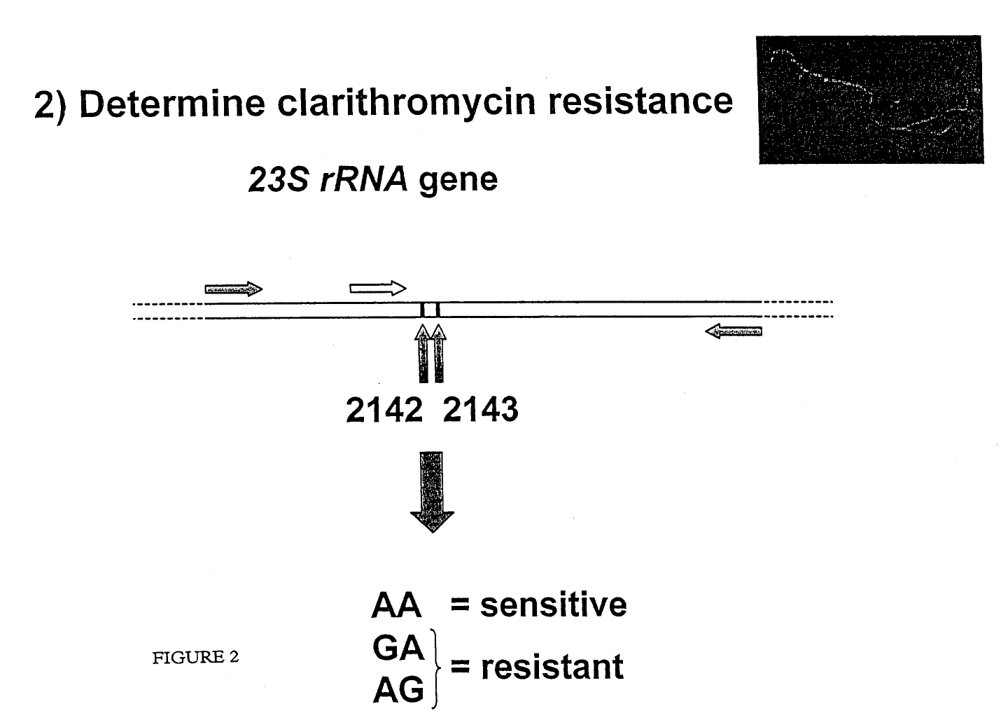

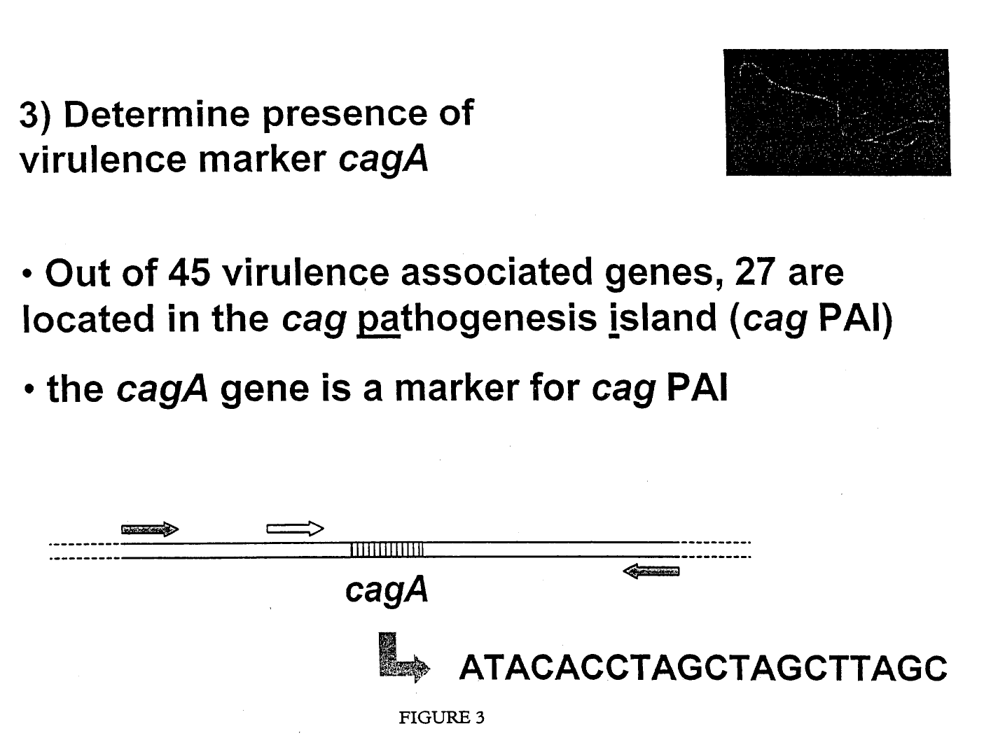

Resistance of H. pylori to the antibiotic clarithromycin is conferred by point mutations in the 23S rRNA gene at positions 2142 (A2142G) and 2143 (A2143G and A2143C). The clarithromycin-resistance markers at 2142 and 2143 of the 23S rRNA gene were analyzed, see FIG. 2.

H. pylori strains vary in their ability to provoke mucosal immune response and epithelial damage. Bacterial virulence is highly associated with the gene products of a 40 kilobase pathogenesis island (cag PAI) (Censini, S. et al. cag, a pathogenicity island of Helicobacter pylori, encodes type I-specific and disease-associated virulence factors. Proc Natl Acad Sci USA 93, 14648-53. (1996)). This cassette of 31 genes, among which is the cytotoxin associated gene A (cag A), is horizontally transferred and present in approximately 60% of H. pylori strains (Covacci, A. et al. Molecular characterization of the 128-kDa immunodominant antigen of Helicobacter pylori associated with cytotoxicity and duodenal ulcer. Proc Natl Acad Sci USA 90, 5791-5. (1993)). Infections with Cag PAI positive strains are associated with a higher production of interleukin-8, and a higher risk of peptic ulcer disease, atrophic gastritis and gastric cancer than infections with Cag PAI negative strains (Blaser, M. J. Role of vacA and the Cag A locus of Helicobacter pylori in human disease. Aliment Pharmacol Ther 10 Suppl 1, 73-7. (1996)). The function of all the genes contained in the Cag PAI is not fully understood, however some of the genes show sequence similarities to a type IV secretion system. It has been shown that the H. pylori system works to translocate Cag A from bacterial cells into gastric epithelial cells, where the protein is phosphorylated and acts to rearrange the cytoskeleton (Odenbreit, S. et al. Translocation of Helicobacter pylori Cag A into gastric epithelial cells by type IV secretion. Science 287, 1497-500. (2000) and Stein, M., Rappuoli, R. & Covacci, A. Tyrosine phosphorylation of the Helicobacter pylori Cag A antigen after cag-driven host cell translocation. Proc Natl Acad Sci USA 97, 1263-8. (2000)). Thus, although its function is not completely known, the cag A gene serves as a genotypic marker for highly virulent H. pylori strains. In Examples 1 and 3, gastric biopsies were tested for the presence of virulence marker gene cagA.

Host genetic factors, together with bacterial, dietary and environmental factors, affect the clinical outcome of H. pylori infections. HLA genotype, blood group antigens and host gastric acid physiology have been implicated to affect the final clinical outcome. Interleukin-1-beta (IL-1B) polymorphisms at positions −511, −31 and +3954 are thought to enhance production of IL-1 and to increase the risk of gastric cancer induced by H. pylori. Patients who are infected with H. pylori, who have low acid secretion and who have relatives with gastric cancer, appear to have a higher frequency of the T-T haplotype of IL-1B −31 and IL-1B −511. IL-1B+3954 T homozygocity was reported to be protective against gastric cancer. A C to T transition at IL-1B −511 and −31 are thus related with increased risk of developing gastric cancer having been infected with H. pylori. In Example 1, all assays were performed on the same gastric biopsy sample.

The invention will now be described by way of non-limiting examples with reference to the drawings in which:

### EXAMPLE 1

Methods & Methods

DNA was isolated from gastric biopsies, or from bacteria grown in liquid and/or solid media, using the DNeasy Tissue kit (Qiagen GmbH, Hilden Germany) according to the manufacturer's instructions. Primers matching highly conserved regions in the 16S rRNA, 23S rRNA and cagA genes were designed to amplify a 133, 184 and 127 bp PCR fragment, respectively. For SNP analysis of genomic DNA from gastric biopsies a 152 bp fragment covering IL-1B+3954 was amplified using standard PCR. For IL-1B −511 and IL-1B −31 a semi-nested approach starting with a touchdown PCR was used. One of the primers for each PCR fragment was biotinylated.

Sample Preparation for Pyrosequencing™

Biotinylated PCR products were immobilized to streptavidin-coated beads (Streptavidin Sepharose™ HP, Amersham Pharmacia Biotech AB, Sweden) using solutions from the PSQ™ 96 Sample Preparation Kit (Pyrosequencing AB) and following a standard protocol. Gel slurry (4 μl) was diluted in binding buffer with PCR product and incubated for 10 minutes at room temperature, mixing continuously. The beads were transferred to a filter plate and the liquid was removed by vacuum filtration (MultiScreen™ Resist Vacuum Manifold, Millipore Inc.). DNA strands were separated in denaturation solution for 1 minute. The immobilized template was washed with washing buffer and then transferred to a PSQ 96 SQA Plate and annealed with 16 pmoles of sequencing primer in 40 μl annealing buffer at 60 C for 5 minutes, followed by cooling at room temperature. For SNP analysis biotinylated PCR products were immobilized to streptavidin-coated magnetic beads (Dynabeads™, Dynal) using a standard protocol. Streptavidin Sepharose HP can be used as an alternative to the magnetic beads. For the IL-1B −511 sample, single-stranded DNA binding protein (2 μg) was added after cooling to room temperature to facilitate the resolution of the pyrogram™.

Pyrosequencing™

Samples were analyzed using a PSQ 96 System together with SQA Software and SQA Reagent Kits (Pyrosequencing AB) for sequence analysis or SNP Software and SNP Reagent Kits for SNP analysis. Standard instructions were followed.

Results

Identification

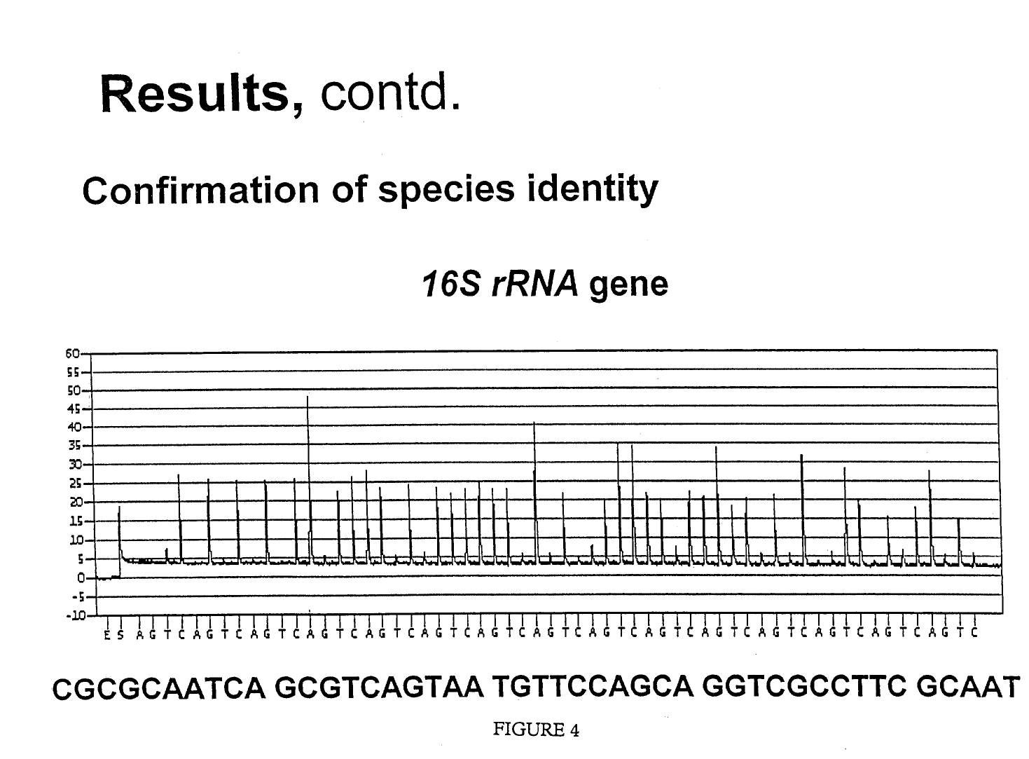

FIG. 4 shows a representative result from the analyses of the 16S rRNA gene from H. pylori. The species identity could clearly be established by the species signature sequence (GCGCAATCAGCGTCAGT) In this study, SQA Software was used to analyze the 17 nucleotides sequenced in 131 isolates. Two samples failed due to poor PCR preparation. Of the remaining 129 isolates (equivalent to a total of 2322 called bases), 126 were read correctly and three failed due to a single base being read incorrectly. This gives an overall accuracy of 99.87%.

The H. pylori signature sequence made it possible to distinguish the organism from a set of other bacterial species (Table 1, Example 2). Isolates containing H pylori were correctly identified and the results were confirmed by biochemical typing.

Characterization

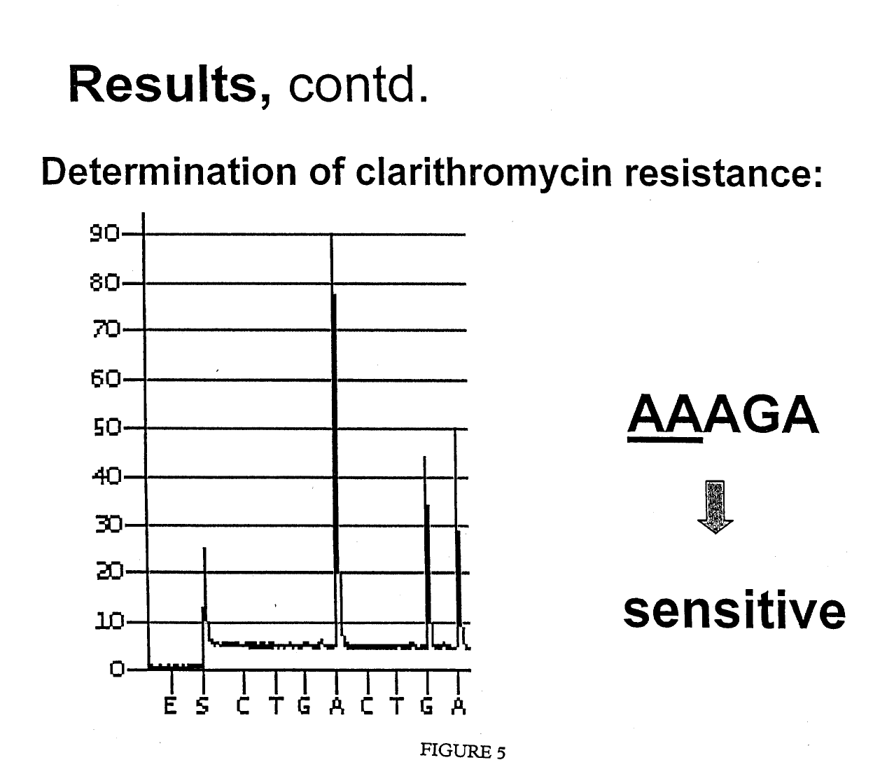

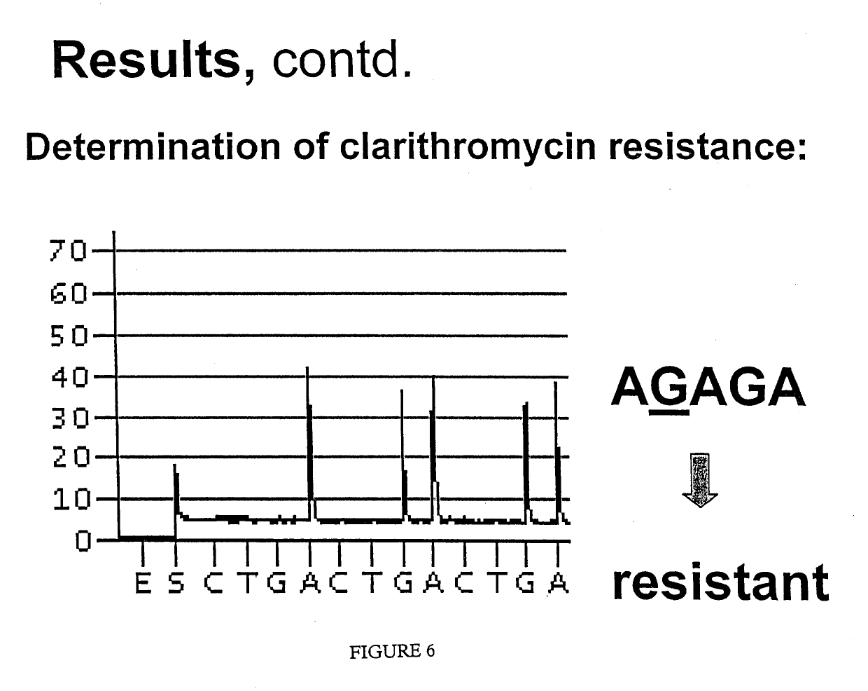

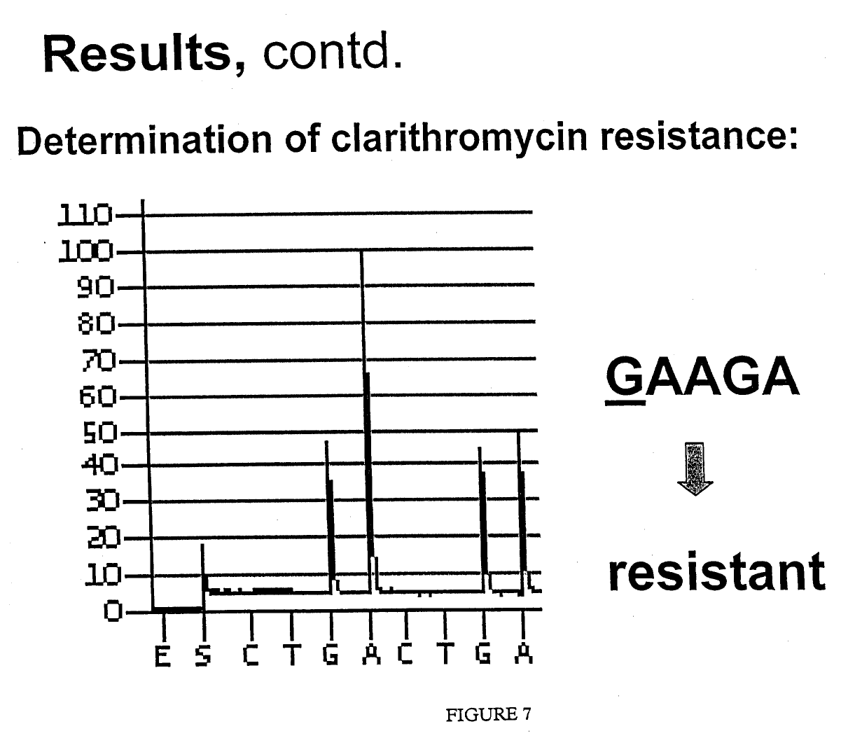

The H. pylori isolates were also tested for clarithromycin resistance by sequence analysis of the 23S rRNA gene. The results representing a wildtype sequence and A to G transitions at positions 2142 and 2143, respectively, are shown in FIGS. 5, 6 and 7. The A2143C mutation was not found in any of the isolates. For 5 or more nucleotides, covering the mutation sites, a total of 154 isolates were correctly genotyped. The results were confirmed by an E-test of minimal inhibitory concentration of clarithromycin.

Patient Genotyping and Characterization of H. pylori in Gastric Biopsies

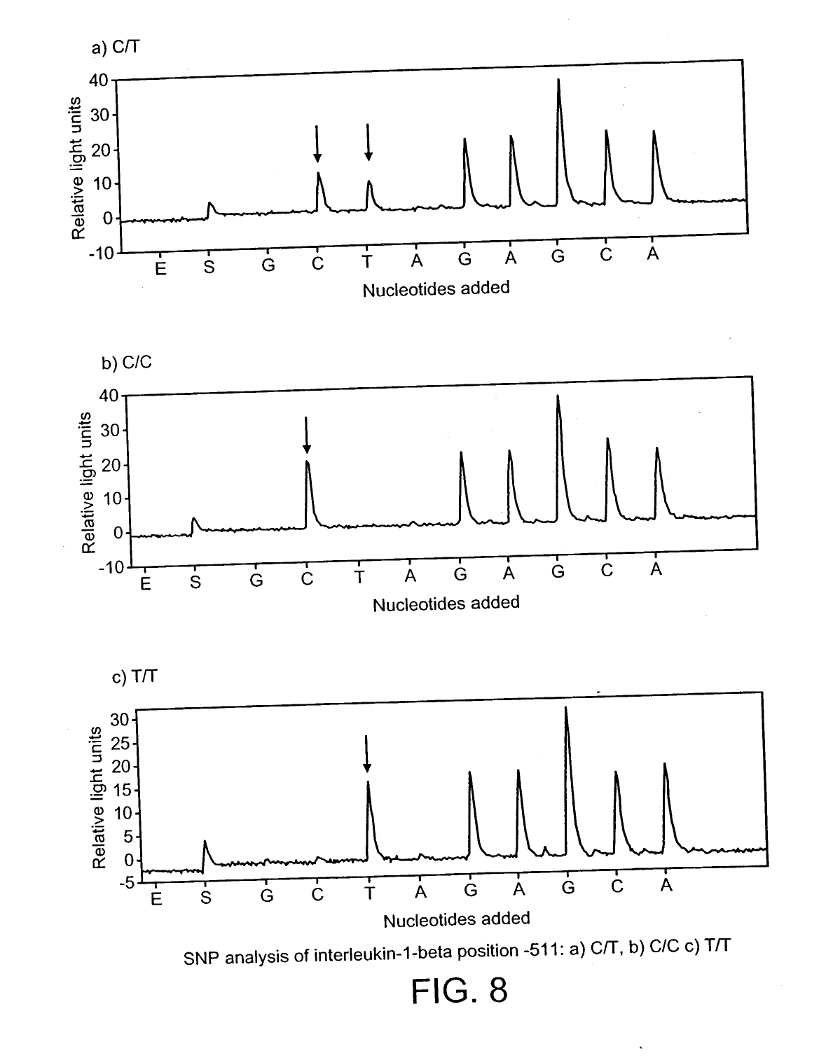

Table 2 (Example 2) shows SNP genotypes from gastric biopsies of interleukin-1-beta −511, −31 and +3954. The three different genotypes were easily assigned. Pyrograms for IL-1B −511 are shown in FIG. 8. By Pyrosequencing, the biopsies were shown to contain H. pylori, a finding confirmed by culture. The isolates were characterized with regard to clarithromycin resistance. To further characterise the bacterial Cag A status, experiments were performed by investigating the presence of a PCR product, verified by Pyrosequencing™ (data not shown). (see Example 3)

Discussion

We used Pyrosequencing technology to rapidly differentiate H. pylori from a number of related species, and to confirm the H. pylori species identity of bacterial isolates. We also assessed the presence of clarithromycin resistance mutations in the same isolates. In gastric biopsies obtained from H. pylori-infected patients, we included an analysis of the virulence status of the isolates by determining the cag A status and host susceptibility factors consisting of three IL-1B gene SNPs. We found that the Pyrosequencing technology produced highly accurate results. In the H. pylori species confirmation assay, 99.87% out of 2322 called bases were accurately read.

### EXAMPLE 2

Identification and Characterisation of Markers in H. pylori Together With Host Susceptibility Markers

Methods

Bacteria were grown in liquid media and DNA was extracted using the Amplicor Respiratory Specimen preparation kit (Roche Diagnostic Systems, Branchburg, N.J., USA). PCR primers matching highly conserved regions in the 16S and 23S rRNA genes were designed to amplify the 133 and 184 bp PCR fragment, respectively. For SNP analysis of genomic DNA from gastric biopsies a 152 bp fragment covering IL-1B+3954 was amplified using standard PCR. For IL-1B −511 and IL-1B −31 a semi-nested approach starting with a touchdown PCR was used. One of the primers for each PCR fragment was biotinylated.

Preparation of Single-Stranded DNA

Biotinylated PCR products were immobilized to streptavidin-coated beads (Streptavidin Sepharose HP, Amersham Pharmacia Biotech AB, Sweden) according to a standard protocol. Gel slurry (4 μl) was filtered and the beads were diluted in binding buffer with 5 pmoles of PCR product and incubated under rotation for 10 minutes at 25 C. The beads were transferred to a filter plate and the liquid was removed by vacuum filtration (Multiscreen Resist Vacuum Manifold, Millipore Inc.). DNA strands were separated by denaturation in 0.2 M NaOH for 1 minute. After washing, the immobilized template was transferred to a PSQ™ 96 SQA Plate and annealed with 16 pmoles of sequencing primer in 40 μl annealing buffer at 60 C for 5 minutes followed by cooling at room temperature.

For SNP analysis a standard protocol using magnetic Dynabeads™ (Dynal AS, Norway) was used for IL-1B −511 2 μg SSB (single-stranded DNA binding protein) was added after primer annealing.

Pyrosequencing™

Samples were analyzed using a PSQ 96 System together with SQA Software and SQA Reagent Kits (Pyrosequencing AB) for sequence analysis or SNP Software and SNP Reagent Kits for SNP analysis. Standard instructions were followed.

Results

Identification

FIG. 4 shows a representative result from the analyses of the 16S rRNA gene from Helicobacter pylori. The species identity could clearly be established by the 17 bp species signature sequence (ACTGACGCTGATTGCGC). SQA Software accurately analyzed the sequence for 18 nucleotides in 126 (96.2%) of the 131 different isolates.

The H. pylori signature sequence made it possible to distinguish the organism from a set of other bacterial species (Table 1). Isolates containing H. pylori were correctly identified and the results were confirmed by biochemical typing.

TABLE 1
Helicobacter pylori distinguished from
six other bacterial species by analysis of 16S rRNA gene.

| Species | 16S rRNA sequence^{a} |
|---|---|
| Helicobacter pylori | ACTGACGCTGATTGCGC |
| Campylobacter upsaliensis | ACTGACGCTAAGGCGGG |
| CCUG 14913 | |
| Campylobacter curvus | ACTGACGCTAATGCGTC |
| CCUG 13146 | |
| Helicobacter cinaedi | ACTGACGCTGATGCGCG |
| CCUG 19218 | |
| Campylobacter hyointestinalis | ACTGACGCTAATGCGTG |
| CCUG 14169 | |
| Helicobacter mustelae | ACTGACGCTGATGCGCG |
| CCUG 25715 | |
| Campylobacter jejuni | ACTGACGCTAAGGCGCG |
| CCUG 11284 | |

^{a}Nucleotide positions 710-726 according to Eckloff et al. (Int J Syst Bacteriol 44: 320-323, 1994)

Characterization

The H. pylori isolates were also tested for clarithromycin resistance by sequence analysis of the 23S rRNA gene. The three possible results are presented in FIGS. 5, 6 and 7. For 5 or more nucleotides, covering the mutation site (A2142G and A2143G), a total of 154 isolates were correctly genotyped. The results were confirmed by an E-test of minimal inhibitory concentration of clarithromycin.

Patient Genotyping

Table 2 shows SNP genotypes from gastric biopsies of interleukin-1-beta −511, −31 and +3954. The three different genotypes were easily assigned. Pyrograms for IL-1B −511 are shown in FIG. 8.

Table 2. SNP genotypes of interleukin-1-beta in gastric biopsies

| | Genotype | Genotype | Genotype |
|---|---|---|---|
| Template | −511 | −31 | +3954 |
| K39 | C/C | T/T | C/T |
| K40 | T/T | C/C | C/T |
| K42 | C/C | T/T | T/T |
| K43 | T/T | C/C | C/T |
| K44 | C/C | T/T | C/C |
| K45 | C/C | T/T | C/C |
| K46 | C/T | T/T | C/C |
| K47 | C/C | T/T | C/C |
| K48 | C/T | C/T | C/T |
| K49 | C/C | T/T | C/C |

### EXAMPLE 3

Sequencing of Cag A Gene

Materials and Methods:

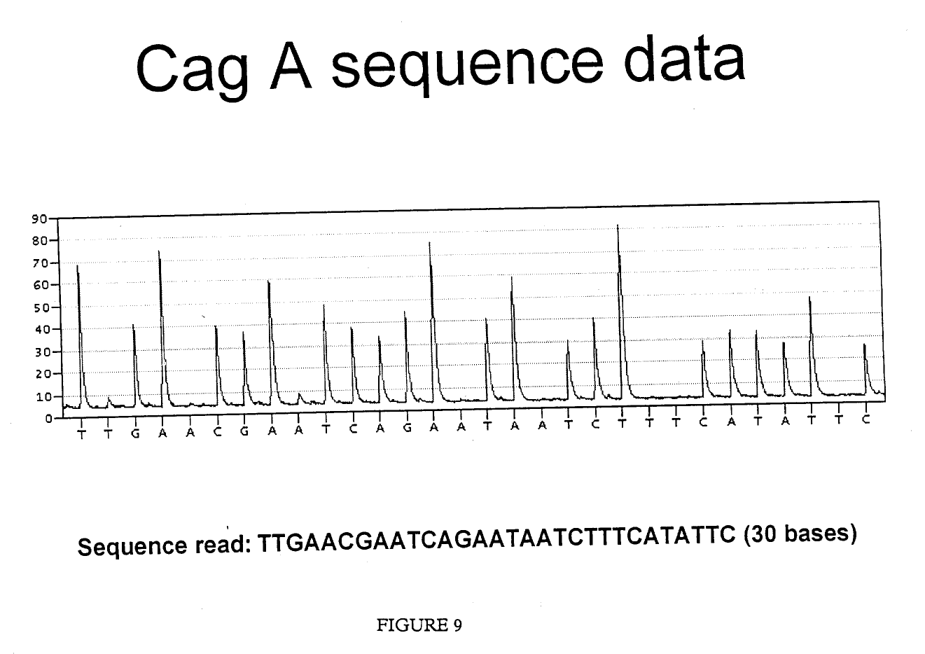

DNA was isolated from gastric biopsies or bacteria grown in liquid or solid media using the DNeasy Tissue kit (Qiagen GmbH, Hilden, Germany) according to the manufacturer's instructions. All reagents used for PCR were purchased from Amersham Biosciences (Uppsala, Sweden), except for primers, which were from Interactiva (Ulm, Germany). A 127-bp PCR fragment was generated using primers matching highly conserved regions in the cagA gene (biotin-5′-AYTAACAGCCACACACGCATT-3′ and 5′-CRGCATTGTTCAACTTGGTG-3′). PCR was performed in 50-μl reaction mixtures containing 1×PCR buffer (10 mM Tris-HCl, 50 mM KCl, 1.5 mM MgCl₂), 1.25 U Taq DNA polymerase, 0.2 mM of each nucleotide, 0.1 μM of each primer and 5 μl eluate containing DNA. The reaction mixture was subjected to 50 cycles of 95 C for 30 seconds, primer annealing at 55 C for 30 s and 72 C for 30 seconds with an initial 5 min denaturation step at 95 C and terminal extension step at 72 C for 10 min. For sequence analysis, biotinylated PCR products were prepared for Pyrosequencing using solutions from the PSQ™ 96 Sample Preparation Kit (Pyrosequencing AB, Uppsala, Sweden). Standard instructions were followed. The sequencing primer used had the sequence GTTTTCACCAAGT. Samples were analyzed using a PSQ 96 System together with SQA Software and SQA Reagent Kits (Pyrosequencing AB). The resulting pyrogram™ showing Cag A sequence data is presented as FIG. 9.

The sequence of the Cag A gene, the primer squence is underlined:

```
cag A_F(2)
AYTAACAGCCACACACGCATTAATAGCATTGTCCAAACTGGAACAATCAATGAAAAAGCGACCGGTATGCTA
ACGCAAAAAAACCCTGAGTGGCTCAAGCTCGTGAATGATAAGATAGTTGCACATAATGTGGGAAGCGCTCAT
TTGTCAGAGTATGATAAAATTGGATTCAACCAAAAGAATATGAAAGATTATTCTGATTCGTTCAAGTTTTCC
ACCAAGTTGAACAATGCCG
                GTGGTTCAACTTGTTACRG
                            cag A_R(2)b
```

Thus, the presence of the Cag A sequence, as depicted in FIG. 9, indicates the virulence of the H. pylori strain present.

1. A method of determining the likely clinical outcome of a microbiological infection in a patient comprising:
(a) analysing a sample taken from said patient for the presence of a target micro-organism, by the characterisation of a target nucleic acid sequence therein; and
(b) analysing a sample taken from said patient for the presence of one or more disease susceptibility markers in the genome of said patient.

2. The method of claim 1 wherein the disease susceptibility marker is a marker associated with, or pre-disposing to, disease, or a genetic or physiological factor or feature associated with, or predisposing to, or being a risk factor for a disease, condition, syndrome or illness.

3. The method of claim 1 further comprising the step of (c) analysing a sample taken from said patient for the presence of one or more drug resistance markers in the genome of the target micro-organism.

4. The method of claim 3 further comprising the step of (d) analysing a sample taken from said patient for the presence of one or more virulence markers in the genome of the target micro-organism.

5. The method of claim 1 wherein the analysis is performed on the same single sample taken from said patient.

6. The method of claim 1 wherein the target nucleic acid sequence of said target micro-organism is the 16s rRNA gene or the RNase P gene.

7. The method of claim 1 wherein the nucleic acid of the micro-organism is characterised by sequencing.

8. The method of claim 7 wherein the sequencing is performed by sequencing by synthesis.

9. The method of claim 1 wherein the genome of said patient is analysed by sequencing.

10. The method of claim 9 wherein the sequencing is performed by sequencing by synthesis.

11. The method of claim 8 wherein the sequencing-by-synthesis is a pyrophosphate detection-based method.

12. A method of selecting a suitable therapeutic regimen for a patient comprising:
(a) analysing a sample taken from said patient for the presence of a target micro-organism, by the characterisation of a target nucleic acid sequence therein; and
(b) analysing a sample taken from said patient for the presence of one or more disease susceptibility markers in the genome of said patient.

13. The method of claim 12 wherein the disease susceptibility marker is a marker associated with, or pre-disposing to, disease, or a genetic or physiological factor or feature associated with, or predisposing to, or being a risk factor for a disease, condition, syndrome or illness.

14. The method of claim 12 further comprising the step of (c) analysing a sample taken from said patient for the presence of one or more drug resistance markers in the genome of the target micro-organism.

15. The method of claim 12 further comprising the step of (d) analysing a sample taken from said patient for the presence of one or more virulence markers in the genome of the target micro-organism.

16. The method of claim 12 wherein the analysis is performed on the same single sample taken from said patient.

17. The method of claim 12 wherein the target nucleic acid sequence of said target micro-organism is the 16s rRNA gene or the RNase P gene.

18. The method of claim 12 wherein the nucleic acid of the micro-organism is characterised by sequencing.

19. The method of claim 18 wherein the sequencing is performed by sequencing by synthesis.

20. The method of claim 12 wherein the genome of said patient is analysed by sequencing.

21. The method of claim 20 wherein the sequencing is performed by sequencing by synthesis.

22. The method of claim 18 wherein the sequencing-by-synthesis is a pyrophosphate detection-based method.

23. A method of determining the likely clinical outcome of infection by Helicobacter pylori in a patient comprising:
(a) analysing a sample taken from said patient for the presence of H. pylori, by the characterisation of a target nucleic acid sequence therein; and
(b) analysing a sample taken from said patient for the presence of one or more disease susceptibility markers in the genome of said patient;
(c) analysing a sample taken from said patient for the presence of one or more drug resistance markers to common antibiotics in the genome of H. pylori;
(d) analysing a sample taken from said patient for the presence of one or more virulence markers in the genome of H. pylori.

24. The method of any one of claims 23, wherein said target microorganism is Helicobacter pylori, said target nucleic acid is the 16s rRNA gene and said disease susceptibility marker is one or more of HLA genotype, blood group antigens, host gastric acid physiology and/or interleukin-1-beta polymorphisms at positions 511, −31 and +3954.

25. The method of claim 23 wherein the drug resistance markers are point mutations in the 23s rRNA gene at positions 2142 and 2143.

26. The method of claim 23 wherein the virulence marker is the Cag A gene.
